(19) United States
(12) Patent Application Publication
(10) Pub. No.: US 2025/0034631 A1
(43) Pub. Date: Jan. 30, 2025

(54) SYSTEMS AND METHODS FOR NUCLEIC ACID EXTRACTION AND VISUAL DETECTION

(71) Applicant: UNIVERSITY OF CONNECTICUT, Farmington, CT (US)

(72) Inventors: Changchun LIU, Farmington, CT (US); Xiong DING, Farmington, CT (US)

(21) Appl. No.: 18/710,724

(22) PCT Filed: Nov. 16, 2022
(86) PCT No.: PCT/US2022/079923

§ 371 Date: May 16, 2024

Related U.S. Application Data
(60) Provisional application No. 63/281,458, filed on Nov. 19, 2021.

(51) Int. Cl. C12Q 1/6844; C12Q 1/6806; C12Q 1/70; G01N 21/77; G01N 21/80

(52) U.S. Cl. CPC C12Q 1/6844; C12Q 1/6806; C12Q 1/701; G01N 21/80; G01N 2021/7759

(57) ABSTRACT

Disclosed are methods, devices and kits for extracting, amplifying, detecting and visualizing nucleic acids of interest for use in onsite rapid diagnostics.

### CROSS REFERENCES

This application claim priority to U.S. Provisional Patent Application Ser. No. 63/281,458, filed Nov. 19, 2021, incorporated by reference here in its entirety.

### STATEMENT OF GOVERNMENT SUPPORT

This invention was made with government support under Grant No. R01EB023607 awarded by the National Institutes of Health. The government has certain rights in the invention.

### SEQUENCE LISTING STATEMENT

A computer readable form of the Sequence Listing is filed with this application by electronic submission and is incorporated into this application by reference in its entirety. The Sequence Listing is contained in the file created on Oct. 13, 2022 having the file name “21-1437-WO.xml” and is 10 kb in size.

### BACKGROUND

Public health has been challenged in recent decades owing to a number of infectious diseases caused by various viruses such as human immunodeficiency virus (HIV), severe acute respiratory syndrome coronavirus (SARS-CoV), influenza A virus subtypes H1N1 and H7N9, Middle East respiratory syndrome coronavirus (MERS-CoV), Zika virus, Ebola virus, and currently emerging severe acute respiratory syndrome coronavirus 2 (SARS-CoV-2). Because of economic globalization, this public health challenge can easily trigger epidemic or even pandemic diseases.

To combat the spread of infectious disease, reverse transcription quantitative polymerase chain reaction (RT-qPCR) is widely used as the gold standard for infection detection. RT-qPCR-based RNA testing can give rapid, sensitive and specific diagnosis, in comparison with other molecular diagnostics such as antigen testing and serological testing of IgM and IgG. However, RT-qPCR methods greatly depend on well-trained personnel, large and expensive detection instrument, and long reaction assay time, which are not adapted to onsite COVID-19 rapid testing and the employment in resource-limited settings. As the alternatives to RT-qPCR assays, isothermal amplification assays such as reverse transcription loop-mediated isothermal amplification (RT-LAMP), reverse transcription recombinase polymerase amplification (RT-RPA), and reverse transcription dual-priming mediated isothermal amplification (RT-DAMP), have been developed for onsite infection testing, because of shorter testing time, independence of large instrument, and visual detection of products. Yet, these approaches so far still require time-consuming nucleic acid extraction, showing long sample-to-answer testing time.

Currently, the main materials for nucleic acid extraction are silica gel membranes and surface-functionalized paramagnetic beads. In these systems, large bench top centrifuges, magnet rods or devices, as well as multiple processes of washing, drying, and eluting nucleic acids are required, which are too complicated for onsite pathogen detection. To eliminate the centrifuging operation and simplify the extraction steps, some paper materials, for example, the cellulose-based flinders technology associates (FTA) cards are developed. However, relatively complex procedures such as punching, washing, and drying the card from sample areas are still indispensable in FTA-based nucleic acid extraction. In addition, the FTA card is only used to archive nucleic acids, not able to mediate visual detection as an indicator. Thus, new functional materials to undertake both nucleic acid extraction and visual detection are desired in order to allow for cost effective and simple onsite rapid diagnostics.

### SUMMARY DISCLOSURE

In a first aspect, the disclosure provides a method of detecting a nucleic acid of interest comprising amplifying by isothermal amplification a nucleic acid of interest present in a nucleic acid sample bound in cellulose fibers of a pH sensitive element to generate an amplification product, wherein the isothermal amplification is performed in the absence of a pH buffer; and wherein the isothermal amplification of the nucleic acid of interest results in the release of hydrogen ions on the pH sensitive element; and identifying a pH detected by the pH sensitive element, wherein a pH of 7.5 or less indicates the release of hydrogen ions during the isothermal amplification and the presence of the nucleic acid of interest in the nucleic acid sample.

In a second aspect, the disclosure provides a device comprising a housing having a top portion and a bottom portion, wherein the top portion of the housing includes a first through-hole; a fluid inlet extending vertically from a top surface of the top portion of the housing, wherein the fluid inlet is in fluid communication with the first through-hole; a non-absorbent layer positioned adjacent a bottom surface of the top portion of the housing, wherein the non-absorbent layer includes a second through-hole vertically aligned with the first through-hole; one or more absorbent layers positioned between the non-absorbent layer and the bottom portion of the housing; wherein the one or more absorbent layers and the non-absorbent layer are positioned to accommodate placement of a pH sensitive element positioned between the one or more absorbent layers and the non-absorbent layer; and wherein a pH sensitive element positioned between the one or more absorbent layers and the non-absorbent layer is vertically aligned with both the first through-hole and the second through-hole.

In a third aspect, the disclosure provides a kit comprising two or more of the following: a pH sensitive element; primers specific for a nucleic acid of interest; reagents for performing isothermal amplification in the absence of a pH buffer; and means of warming the isothermal amplification reagents.

### BRIEF DESCRIPTION OF FIGURES

FIG. 1 shows a scheme of instrument-free SARS-CoV-2 rapid detection enabled by pH-paper-based RNA extraction and visual detection (termed pH-EVD).

FIG. 2 shows fabrication and characterization of non-bleeding pH-paper based extraction system. A. The assembly of pH-paper extraction system in a 3D-printed device. B. The time course of liquid flow for three time of sequential loading into the system. Dark liquid was used for easy illustration. C. SEM images of partial pH paper after RNA extraction.

FIG. 3 shows visual isothermal amplification by directly inserting non-bleeding pH paper into reaction solutions. A. shows the schematic illustration of RT-DAMP. B. shows hydrogen ions produced during primer extension. C. shows the time course of pH paper's color change as reaction goes on and the EvaGreen™-based real-time fluorescence detection. Positive, the reactions with 6.8×10⁴ copies of SARS-CoV-2 RNA. NTC, the reactions without any template. D. shows the specificities of pH-paper-based visual detection and the real-time fluorescence detection. The SARS-CoV-2 PC, SARS-CoV control, MERS-CoV control, and Hs_RPP30 PC were all commercialized plasmids with corresponding gene sequences (Integrated DNA Technologies IDT). E. shows the sensitivities of pH-paper-based visual detection and the real-time fluorescence detection. Various copies of commercialized SARS-CoV-2 RNA control from Twist Bioscience were used. The visual detection was conducted with 40 min incubation.

FIG. 4 shows pH-paper-based RNA extraction for visual isothermal amplification detection. A. shows the effect of various diameters of pH paper on extraction performance. In this experiment, 28 μL sample, 112 μL Buffer AVL, 112 μL absolute ethyl alcohol, 112 μL Buffer AW1, and 112 μL Buffer AW2 from the commercial QIAamp™ Viral RNA Mini Kit (QIAGEN) were used. Buffer AVL is a lysis buffer. Buffer AW1 and AW2 are two washing buffers. B. shows the effect of various amounts of the extraction reagents on extraction performance. The volume's ratio of Buffer AVL, absolute ethyl alcohol, Buffer AW1, and Buffer AW2 strictly follows the kit's instructions. Heat-inactivated SARS-CoV-2 with 1.2×10⁵ or 1.2×10² genome equivalents (GE) per microliter (GE μL⁻¹) in 1× phosphate-buffered saline (PBS) is the sample provided by BEI Resources.

FIG. 5 shows the performance of pH-paper-based extraction and visual detection (termed pH-EVD) on testing various amounts of heat-inactivated SARS-CoV-2. A. shows the procedures of the pH-EVD assay and the parallel assays including direct pH-paper-based visual detection, extraction-free colorimetric RT-DAMP using cresol red, and extraction-free TaqManr™ probe-based RT-qPCR. B. shows the comparison of visual detection results. C. shows the percent positives for various assays. Three independent experiments were investigated for each assay. All the visual detections were incubated for 40 min. GE, genome equivalents. NTC, the solutions without any heat-inactivated SARS-CoV-2.

FIG. 6 shows the performance of pH-EVD on testing various amounts of heat-inactivated SARS-CoV-2 spiked in human saliva. A. shows the procedures of the pH-EVD assays with lysate and heat treatment of saliva, and the parallel assays with heat-treated saliva for direct pH-paper-based visual detection, extraction-free colorimetric RT-DAMP using cresol red, and extraction-free TaqMan™ probe-based RT-qPCR. B. shows the comparison of visual detection results. C. shows the percent positives for various assays. Three independent experiments were investigated for each assay. All the visual detections were incubated for 40 min. GE, genome equivalents. NTC, the solutions without any heat-inactivated SARS-CoV-2.

FIG. 7 shows the clinical validation of instrument-free SARS-CoV-2 rapid detection enabled by pH-EVD. A. shows the procedures of the instrument-free detection and the routine RT-qPCR assay on testing clinical NP samples and contrived saliva samples. B. and C. show the Cq values of the 33 saliva samples and 30 clinical NP samples by RT-qPCR following spin column-based extraction, respectively. D. and E. shows the visual detection results of the instrument-free rapid detection (pH-EVD) for the saliva and NP samples, respectively. F. shows the confusion matrix describing the overall performances of the two assays between positive and negative samples. The spin column-based extraction and RT-qPCR are considered as the standards. The samples indicated by dark color are tested to be negatives by pH-EVD, while positives by RT-qPCR.

FIG. 8 shows the real-time fluorescence curves of A. RT-qPCR for testing various concentrations of heat-inactivated SARS-CoV-2 and B. the linear relationship between Cq and the logarithm with base 10 of target concentrations. GE, genome equivalents. NTC, the solutions without any heat-inactivated SARS-CoV-2. Three replicates were run (n=3). The Cq value indicated was the averaged with the standard deviation of the three replicates.

FIG. 9 shows the real-time fluorescence curves of A. RT-qPCR for testing contrived 90% (v/v) saliva solutions containing 10% (v/v) of heat-inactivated SARS-CoV-2 and B. the linear relationship between Cq and the logarithm with base 10 of target concentrations. GE, genome equivalents. NTC, the solutions without any heat-inactivated SARS-CoV-2. Three replicates were run (n=3). The Cq value indicated was the averaged with the standard deviation of the three replicates.

FIG. 10 shows the handheld, chemically-heated, smart cup used for instrument-free SARS-CoV-2 detection. A. shows the photograph of the smart cup. B. shows the photograph of 3D-printed metal tube holder.

FIG. 11 shows the cost comparison of A. instrument-free SARS-CoV-2 rapid detection with pH-EVD and the B. RT-qPCR with spin column-based RNA extraction. The cost only focuses on the commercially available materials and reagents used in this study, not covering equipment fees and staff costs.

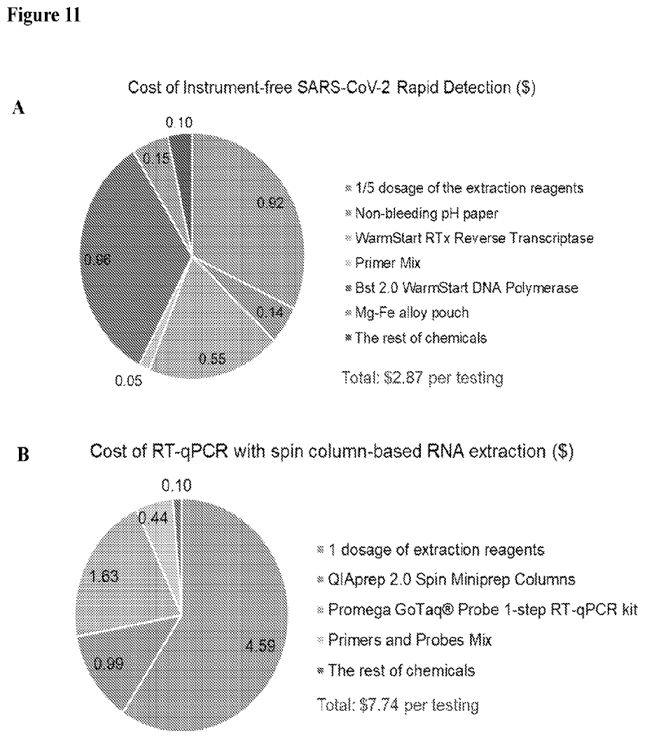

FIG. 12 shows the 3D-printed device used for pH-paper-based extraction.

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology. Vol. 185, edited by D. Goeddel, 1991. Academic Press, San Diego, CA), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2nd Ed. (R. I. Freshney. 1987. Liss, Inc. New York, NY), Gene Transfer and Expression Protocols, pp. 109-128, ed. E. J. Murray, The Humana Press Inc., Clifton, N.J.), and the Ambion 1998 Catalog (Ambion, Austin, TX).

As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gin; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Additionally, the words “herein,” “above.” and “below” and words of similar import, when used in this application, shall refer to this application as a whole and not to any particular portions of the application.

In a first aspect, the disclosure provides a method of detecting a nucleic acid of interest comprising amplifying by isothermal amplification a nucleic acid of interest present in a nucleic acid sample bound in cellulose fibers of a pH sensitive element to generate an amplification product, wherein the isothermal amplification is performed in the absence of a pH buffer; and wherein the isothermal amplification of the nucleic acid of interest results in the release of hydrogen ions on the pH sensitive element; and identifying a pH detected by the pH sensitive element, wherein a pH of 7.5 or less indicates the release of hydrogen ions during the isothermal amplification and the presence of the nucleic acid of interest in the nucleic acid sample.

As used herein, pH sensitive element binds a nucleic acid sample by binding the nucleic acids to the cellulose fibers of the filter and is capable of detecting pH levels. According to the methods of the invention, the isothermal amplification for a nucleic acid of interest is performed on the pH sensitive paper. If the nucleic acid of interest is present in the nucleic acid sample, the nucleic acid of interest is amplified by the isothermal amplification. The amplification of a nucleic acid of interest results in the release of hydrogen ions onto the pH sensitive element.

According to the methods of the invention, the isothermal amplification is performed in the absence of a pH buffer. The pH is detected by the pH sensitive element after the isothermal amplification. The release of hydrogen ions in the absence of a pH buffer results in the pH sensitive element detecting a pH of 7.5 or less. If the nucleic acid of interest is not present in the nucleic acid sample, then isothermal amplification will not amplify a nucleic acid and will not release hydrogen ions. In the absence of a release of hydrogen ions the pH sensitive element will detect a pH of more than 7.5. For example, pH of about 7.6, 7.8, 8, 8.2, 8.4, 8.6, 8.8, 9, 9.2, 9.4, 9.6, 9.8, 10, 10.2, 10.4, 10.6, 10.8, 11, 11.2, 11.4, 11.6, 11.8, 12, 12.2, 12.4, 12.6, 12.8, 13, 13.2, 13.4, 13.6, 13.8, and pH of 14.

In various embodiments of the method, prior to the amplifying, the method comprises obtaining the nucleic acid sample from a biological sample, wherein the obtaining comprises lysing cells present in the biological sample to generate a processed biological sample, and contacting the processed biological sample with the pH sensitive element to bind the nucleic acid sample to the cellulose fibers of the pH sensitive element.

The nucleic acid sample can be obtained from any biological sample suitable for use according to the methods of the invention. In biological samples where the nucleic acids are contained within cells or present in bodily fluids, the nucleic acid sample can be obtained by lysing the cells present in a biological sample in order to release the nucleic acids from the cells. This produces a processed biological sample comprising nucleic acids which are free to contact and bind the pH sensitive element.

As used herein, the pH sensitive element comprises cellulose fibers which bind to the nucleic acids of the processed biological sample.

As used herein, a “lysin” is any substance which causes the lysis of a cell. In various embodiments of the method, lysing comprises treating the biological sample with a lysin, exposing the biological sample to heat, or a combination thereof. The lysing of the biological sample can be accomplished using any method of lysis, including, but not limited to exposure to heat, treatment with a lysin, or a combination thereof.

In various embodiments of the method, the contacting step comprises filtering the processed biological sample through the pH sensitive element, wherein the nucleic acid sample from the processed biological sample binds to the pH sensitive element. The contacting of the processed biological sample to the pH sensitive element can comprise filtering the sample through the pH sensitive element.

Filtering of the processed biological sample through the pH sensitive elements comprises passing the processed biological sample through the pH sensitive element. During the filtering of the processed biological sample, the nucleic acid sample binds to the pH sensitive element and results in the extraction of the nucleic acid sample from the unbound components of the processed biological sample. The unbound components can then be removed/discarded prior to the isothermal amplification. The filtration of the processed biological sample through the pH sensitive element can be accomplished by any suitable method including, but not limited to capillary force, gravitational force, applied suction, or a combination thereof. In one, non-limiting embodiment, the filtering comprises filtering the biological sample through the pH sensitive element by capillary force.

Filtering can occur over any amount of time. In one, non-limiting embodiment the filtering is completed in less than 10 minutes. In various other embodiments, the filtering is completed in less than 1, 2, 3, 4, 5, 6, 7, 8, 9, or 10 minutes. In another non-limiting embodiment, the filtering is completed in less than 1 minute.

According to the methods of the invention, the lysing, contacting, or filtering can further comprise one or more wash step(s) and wherein the one or more wash steps occurs prior to the isothermal amplification. The one or more wash steps can remove components of the biological sample which do not bind the pH sensitive element.

According to the method of the invention a pH of between 6 and 7.5 indicates the presence of the nucleic acid of interest in the nucleic acid sample. For example, pH of about 6, 6.1, 6.2, 6.3, 6.4, 6.5, 6.6, 6.7, 6.8, 6.9, 7, 7.1, 7.2, 7.3, 7.4, and 7.5. In various other embodiments, the pH can be less than 6. In one, non-limiting embodiment, a pH of more than 7.5 indicates the absence of the nucleic acid of interest in the nucleic acid sample.

The method of any one of the preceding claims, wherein the nucleic acid sample and/or the nucleic acid of interest is RNA.

The nucleic acid sample and/or the nucleic acid of interest can be any type of nucleic acid including, but not limited to, DNA or RNA.

The type of isothermal amplification can be selected based on the type of nucleic acid sample, biological sample and/or nucleic acid of interest.

According to the methods of the invention, the pH sensitive element can be any suitable type of pH sensitive material, which binds nucleic acids. This includes, but is not limited to, commercially available pH paper. In one-non-limiting embodiment, the pH sensitive element comprises non-bleeding pH paper, widely available from many commercial suppliers including but not limited to Sigma Aldrich. In non-bleeding pH paper, the pH sensitive dyes are bound to the paper, preventing the pH sensitive dyes from bleeding into the sample. This prevents contamination of the sample with the pH sensitive dyes.

According to the methods of the invention, the pH sensitive element can provide any suitable read-out of pH detection. In one non-limiting embodiment, the pH sensitive element detects the pH by colorimetric detection. As used herein, “colorimetric detection” means any read-out that can be detected by a color of the pH sensitive element, including but not limited to colorimetric detection provided for by a wide range of commercially available pH papers. In various other embodiments, the pH sensitive element detects the pH by fluorescent detection.

The isothermal amplification can include any type of isothermal amplification suitable for use according to the methods of the invention, including, but not-limited to dual-priming mediated isothermal amplification (DAMP), reverse transcription DAMP (RT-DAMP), loop-mediated isothermal amplification (LAMP), nucleic acid sequence based amplification (NASBA), recombinase polymerase amplification (RPA), helicase dependent amplification (HDA), rolling circle amplification (RCA), strand-displacement amplification (SDA), exponential amplification reaction (EXPAR), or a combination thereof.

The isothermal amplification can comprise any suitable combination of steps as disclosed in the following: U.S. Pat. No. 7,316,910 (LAMP); CN102534052 (NASBA); WO2008035205 (RPA); U.S. Pat. No. 7,282,328 (HAD); U.S. Pat. No. 6,783,943 (RCA); U.S. Pat. No. 5,712,124 (SDA); and WO2004067726 (EXPAR), all of which are incorporated by reference herein.

In one non-limiting embodiment, the isothermal amplification is DAMP/RT-DAMP and comprises any suitable combination of steps as disclosed in US Publication 20220081712, which is incorporated by reference herein.

As used herein “dual-priming mediated isothermal amplification (DAMP)” is an isothermal nucleic acid amplification method for molecular detection with high sensitivity and specificity. A total of six sequence-specific primers are designed in DAMP, including the forward and reverse outer primers of FO and RO, the forward and reverse inner primers of FI and RI, and the forward and reverse pairing-competition primers of FC and RC. These six primers specifically recognize six distinct sites flanking the F3, F2, R1 sites of the forward target sequence and the R3c, R2c, Fc sites of the target reverse sequence. F and R mean the “forward and reverse” directions, respectively. The lowercase “c” represents “complementary”. For example, the F3c and F3 sites are complementary sequences. Both the outer- and pairing-competition primers are single-site primers. FO (RO) primer uses the F3 (R3c) site and FC (RC) primer employs the R1 (F1c) site. In contrast, the inner primers are double-site primers. FI primer is composed of F1c and F2 sites with a “TTTT” four-thymine spacer, and the F2 site is at the 3′-end. RI primer consists of R1, the “TTTT” spacer, and R2c. The TTTT spacer is introduced into the inner primers to destabilize the primer-dimers.

Compared to conventional LAMP primers design, the DAMP method has two distinct features: i) each inner primer is designed to recognize two target sites with the distance below 40 nt and inserted with a TTTT spacer, which ensures efficient “dual-priming” extension. However, in the LAMP method, the distance between two target sites of the inner primers is recommended to be about 40-60 nt to initiate efficient “self-priming” extension according to the LAMP primer design guide. Second, to accelerate the amplification, two pairing competition primers are added. But the addition of the pairing-competition primers does not increase the total number of target sites and complicates the primer design because their sequences are the same as the 5′-parts of the inner primers (FI/RI).

The DAMP assay typically contains two steps: i) basic structure producing step and ii) cycling amplification step. In the DAMP assay, DNA synthesis is initiated by DNA polymerase when the F2 region of FI primer anneals to the F2c site and the FO primer anneals to the F3c site. Due to the strand-displacement activity of the DNA polymerase, the strand elongated from FI primer is displaced and released by the FO primer extension. Then, R2 and R3 sites in the released strand are recognized by RI and RO primers, respectively. Afterwards, the extended strand by RI primer is displaced by the elongated strand from RO primer. The released strands form basic structure for the downstream cycling amplification. Since the RI primer contains two target sites with a distance of less than 40 nt and a TTTT spacer, the “self-priming” strand extension capability is reduced compared to the dumb-bell structure in conventional LAMP method. Furthermore, due to the addition of pairing competition primers, the weakened “self-priming” extension will fully compete with three “pairing-priming” extension events: i) the first “pairing-priming” extension is initiated by the annealing of F2 site in FI primer to the F2c site, ii) the second one results from the annealing competition of FC primer to the F1 site, and iii) the third one is the concurrence of the aforementioned two events. The “self-priming” extension is able to produce dsDNA fragments with a closed loop which can be recognized by FI primer.

Then, the “self-priming” and “pairing-priming” strand extensions take place at their 3′-end parts again, and the amplification proceeds. Unlike “self-priming” extension, the pairing-priming” extension can simultaneously generate multiple basic structures for cycling amplification, including duplex basic structure and the complementary basic structure. However, only one dumb-bell structure is produced at this step in the conventional LAMP, due to strong “self-priming” but weak “pair-priming” strand extension. Analogously, “self-priming” and “pairing-priming” extensions occur at both ends of the duplex basic structure.

The DAMP method has two distinct features on primers design. First, each inner primer in DAMP is designed to recognize two target sites with the distance below 40 nucleotides (nt) and inserted with a TTTT spacer, which ensures efficient “dual-priming” extension. However, in the LAMP method, the distance between the two target sites used for inner primer design is recommended to be about 40-60 nt to initiate efficient “self-priming” extension according to the LAMP primer design guide. Second, to accelerate the amplification, two pairing competition primers are added. But the addition of the pairing-competition primers does not increase the total number of target sites and complicates the primer design because their sequences are the same as the 5′-parts of the inner primers.

According to the methods of the invention the biological sample can be from any suitable source, including, but not limited to, any mammalian source. In one non-limiting embodiment, the biological sample is a human biological sample. The biological sample can be any biological sample suitable for use according to the methods of the invention, including, but not limited to saliva, mucous, blood, plasma, whole blood, urine, tissue, cerebrospinal fluid, or a combination thereof. In various embodiments, the biological sample is saliva or nasal mucus.

According to the method of the invention, the isothermal amplification can occur in a solid phase or can be performed in a solution. In one embodiment, the isothermal amplification is performed in solution. In another embodiment the isothermal amplification can occur in a cellulose fiber matrix.

In various embodiments, the solution can be any pH prior to isothermal amplification. In one, non-limiting embodiment, the pH of the solution is about 8.8 prior to the isothermal amplification. In another embodiment, the solution is more than 8.0 prior to the isothermal amplification. In still another embodiment, the pH of the solution is between 8.0 and 9.0 prior to the isothermal amplification.

The isothermal amplification can be performed using any method of incubation including both electric and non-electric incubation. In one, non-limiting example, the incubation is achieved using a handheld, non-electric incubator smart cup, which allows the isothermal amplification to occur outside of the laboratory. A smart cup is a handheld incubator which uses chemical reactions to produce the heat to incubate the isothermal amplification reaction.

The methods of the invention can be used to diagnosis the presence of a pathogenic organism. The detection of a nucleic acid of interest which is specific for a particular pathogenic organism indicates presence of the particular pathogenic organism in the sample. The methods of the invention thereby allow for the simple, rapid, and reliable detection of the presence of a pathogenic organism. The pathogenic organism can be any pathogenic organisms including viruses, bacteria, funguses, parasites, protists, or a combination thereof. Non-limiting example of pathogenic organisms include SARS-CoV-2, human immunodeficiency virus (HIV), tuberculosis (TB), ebola, meningitis, influenza A, influenza B, human papilloma virus (HPV), hepatitis C virus (HCV), hepatitis B virus (HBV), or a combination thereof.

The methods of the invention can be carried out without any electric instruments and thus can be carried out outside of the laboratory and at the onsite location where the nucleic acid sample or biological sample is obtained. The methods of the invention are thus not limited to being carried out in any location. In one, non-limiting embodiment, the method is carried out without any instruments requiring electricity.

The isothermal amplification may be carried out at any temperature suitable for a specific use and specific isothermal amplification technique used. In various non-limiting embodiments, the isothermal amplification is carried out at a temperature of between 60° and 65°. In one non-limiting embodiment, the isothermal amplification is carried out at between 55° and 70°.

The isothermal amplification may be carried out for any time suitable for a specific use and specific isothermal amplification technique used. In various non-limiting embodiments, the isothermal amplification is carried out for between 35 and 50 minutes. In one non-limiting embodiment, the isothermal amplification is carried out for between 30 and 60 minutes.

In various non-limiting embodiments, the methods can detect at least 1,200 genome equivalent per microliter. In one non-limiting embodiment, the methods can detect at least 1,000 genome equivalent per microliter. For example, the methods can detect 1,000-1,200 genome equivalent per microliter; 1,000-1,500 genome equivalent per microliter; 1,000-2,000 genome equivalent per microliter; 1,200-1,500 genome equivalent per microliter; 1,500-2,000 genome equivalent per microliter; and/or 500-1.000 genome equivalent per microliter.

In a second aspect, the disclosure provides a device comprising a housing having a top portion and a bottom portion, wherein the top portion of the housing includes a first through-hole; a fluid inlet extending vertically from a top surface of the top portion of the housing, wherein the fluid inlet is in fluid communication with the first through-hole; a non-absorbent layer positioned adjacent a bottom surface of the top portion of the housing, wherein the non-absorbent layer includes a second through-hole vertically aligned with the first through-hole; one or more absorbent layers positioned between the non-absorbent layer and the bottom portion of the housing; wherein the one or more absorbent layers and the non-absorbent layer are positioned to accommodate placement of a pH sensitive element positioned between the one or more absorbent layers and the non-absorbent layer; and wherein a pH sensitive element positioned between the one or more absorbent layers and the non-absorbent layer is vertically aligned with both the first through-hole and the second through-hole.

The non-absorbent layer can be made of any material which does not absorb nucleic acids and the other components of a biological sample. In one non-limiting embodiment the non-absorbent layer can be made of parafilm.

The one or more absorbent layers can be made of any suitable material which absorbs the components of a biological sample including, but not limited to, water, lipids, salts, proteins and enzymes. The number of absorbent layers can be modified to increase the total absorbed volume, wherein additional absorbent layers can be added to absorb more sample volume.

The position of the one or more absorbent layers and the non-absorbent layer are positioned to accommodate the placement of a pH sensitive element of any suitable shape and size.

In one non-limiting embodiment, the device further comprises the pH sensitive element positioned between the one or more absorbent layers and the non-absorbent layer.

According to the invention device, the one or more absorbent layers can comprise an absorbent paper layer and an absorbent pad layer wherein the absorbent paper layer is positioned between the absorbent pad layer the bottom portion of the housing. According to this embodiment of the device, the absorbent paper layer is thinner and absorbs less volume than the absorbent pad layer. The two layers of absorbent material are combined in order to absorb the necessary sample volume.

The pH sensitive element of the device can be any size suitable for use with the device. In one, non-limiting embodiment, the pH sensitive element is the same diameter or larger than the second through-hole. In another embodiment, the pH sensitive element is twice the diameter of the second through-hole. In various other embodiments of the invention the pH sensitive element is between 2 mm and 4 mm and the second through-hole is between 1 mm and 2 mm.

According to the invention device the top portion of the housing can be rotatably coupled to the bottom portion of the housing such that the device is configured to transition from an open configuration to a closed configuration as the top portion of the housing rotates with respect to the bottom portion of the housing.

The fluid inlet can be any suitable size suitable for use with the device. In one non-limiting embodiment, the fluid inlet is greater than a diameter of the first through-hole.

In various embodiments of the device, the bottom surface of the top portion of the housing is substantially flat, and wherein a top surface of the bottom portion of the housing includes a recess configured to receive the non-absorbent layer and the one or more absorbent layers.

In a third aspect, the disclosure provides a kit comprising two or more of the following: a pH sensitive element; primers specific for a nucleic acid of interest; reagents for performing isothermal amplification in the absence of a pH buffer; and means of warming the isothermal amplification reagents.

The pH sensitive element can be any suitable filter which binds nucleic acids in a nucleic acid sample and which is capable of detecting pH. One non-limiting embodiment the pH sensitive element is non-bleeding pH paper. In non-bleeding pH paper, the pH sensitive dyes are bound to the paper, preventing the pH sensitive dyes from bleeding into the sample. This prevents contamination of the sample with the pH sensitive dyes.

The primers specific for a nucleic acid of interest can comprise any primers suitable for use with isothermal amplification. In various embodiments, the primers comprise at least 2, 3, 4, 5, or 6 primers. The primers can include FO, F1, FC, RC, R1 and/or RO primers. In one non-limiting embodiment, the primers comprise SEQ ID NOs: 1-6.

The reagents for performing isothermal amplification can include any reagents suitable for use in performing dual-priming mediated isothermal amplification (DAMP) loop-mediated isothermal amplification (LAMP), nucleic acid sequence based amplification (NASBA), recombinase polymerase amplification (RPA), helicase dependent amplification (HDA), rolling circle amplification (RCA), strand-displacement amplification (SDA), exponential amplification reaction (EXPAR), or a combination thereof in the absence of pH buffers.

The means of warming the isothermal amplification reagents can include any means suitable for use with isothermal amplification, including, but not limited, to electric and non-electric incubators. In one non-limiting embodiment, the means of warming the isothermal amplification reagents is a non-electric handheld incubator, which uses chemical reactions to produce heat.

In various embodiments, the kit can further comprise a positive control and/or a negative control. The positive control and/or the negative control comprise their own pH sensitive elements and reagents for performing isothermal amplification in the absence of a pH buffer.

The positive control and/or the negative can further comprise any other components suitable to serve as controls. In various embodiments, the positive control and/or negative control are in separate containers and are separate from a test container comprising the two or more of a pH sensitive element, primers specific for a nucleic acid of interest, and reagents for performing isothermal amplification in the absence of a pH buffer.

In various embodiments, the kit can further comprise one or more wash buffers. The one or more wash buffers can be any buffer capable of removing components of a nucleic acid sample or biological sample which do not bind the pH sensitive element.

In various embodiments, the kit can further comprise a lysin.

In various embodiments of the kit, the pH sensitive element is non-bleeding pH paper.

In various embodiments of the kit, the primers specific for a nucleic acid of interest can be any primers specific for any nucleic acid of interest. In one non-limiting embodiment, the primers comprise SEQ ID NOs: 1-6.

The kit can comprise any and all reagents suitable for performing isothermal amplification in the absence of a pH buffer. In various embodiments, the kit comprises 1, 2, 3, 4, 5, or all 6 of (NH₄)₂SO₄, Tween® 20, KCl, KOH, dNTPs, and MgSO₄.

In various embodiments the kits can be used to identify nucleic acids of interest for any pathogenic organism. The pathogenic organism can be any pathogenic organisms including viruses, bacteria, funguses, parasites and protists. Non-limiting example of pathogenic organisms include SARS-CoV-2, human immunodeficiency virus (HIV), tuberculosis (TB), ebola, meningitis, influenza A, influenza B, human papilloma virus (HPV), hepatitis C virus (HCV), hepatitis B virus (HBV), or a combination thereof. In one non-limiting embodiment the nucleic acids of interest are specific for SARS-CoV-2.

### EXAMPLES

### Materials and Methods

Materials: MColorpHast pH-indicator strips (non-bleeding; Cat. No. 1.09543.0001) for pH 6.5-10.0, round absorbent pad (AP1004700), betaine (5.0 M), (NH₄)₂SO₄, Tween® 20, KCl, cresol red, and KOH were purchased from MilliporeSigma (Burlington, MA). Parafilm M laboratory wrapping film and absolute ethyl alcohol were purchased from Fisher Scientific (East Greenwich, RI). EvaGreen™ dye (20×) was purchased from Biotium (Fremont, CA). QIAamp™ Viral RNA Mini Kit was purchased from Qiagen (Hilden, Germany). Mg₂SO₄ (100 mM), dNTP mix (10 mM of each), ET SSB (500 μg mL⁻¹), Bst 2.0 WarmStart™ DNA polymerase (Bst 2.0 WS; 8000 U mL⁻¹), WarmStart™ RTx reverse transcriptase (WS RTx; 15,000 U mL⁻¹), and nuclease-free water were purchased from New England BioLabs (Ipswich, MA). GoTaq™ Probe 1-Step RT-qPCR kit was purchased from Promega (Madison, WI). Heat-inactivated SARS-CoV-2 (isolate USA-WA 1/2020) control was purchased from BEI Resources (Manassas, VA). Twist synthetic SARS-CoV-2 RNA control was purchased from Twist Bioscience (South San Francisco, CA). SARS-CoV-2 N positive control (SARS-CoV-2_PC), SARS-CoV control, and Middle East respiratory syndrome coronavirus control (MERS-CoV control), human RPP30 gene control (Hs_RPP30_PC), and all the primers and probes for both CDC-released SARS-CoV-2 N1 RT-qPCR assay and pH-EVD were purchased from Integrated DNA Technologies (Coralville, IA). Pooled human saliva (5.0 mL) was purchased from Innovative Research, Inc. (Novi, MI). A total of 30 clinical NP swab samples and the 33 RNA extracts from clinical NP swab samples were handled in compliance with ethical regulations and the approval of Institutional Review Board of the University of Connecticut Health Center (protocol #: P61067).

RNA Extraction: RNA extraction from heat-inactivated SARS-CoV-2 controls, contrived and clinical samples were conducted by using the pH-paper-based extraction system or spin column-based extraction. For pH-paper-based extraction, a parafilm and an absorbent pad first sandwiched the punch of pH paper. Then, the sandwiched pH paper was placed in a 3D-printed device filled with absorbent paper. After sample collection, 28 μL sample's aliquot was mixed with 112 μL Buffer AVL and 112 μL absolute ethyl alcohol, before loading into the device's container. Sixteen seconds later, 100 μL Wash Buffer 1 was added for the first washing. Afterwards (about 15 s), 100 μL Wash Buffer 2 was added for the second washing. Subsequently, the pH paper was peeled off with a tweezer to get the purified RNA. The Buffer AVL, Wash Buffer 1, and Wash Buffer 2 were all from the QIAamp™ Viral RNA Mini Kit. For spin column-based extraction, the procedure was strictly according to the instruction of the kit. Briefly, 140 μL sample aliquot was mixed with 560 μL Buffer AVL and 560 μL absolute ethyl alcohol, prior to the addition into the spin column, followed by two times washing using 500 μL Wash Buffer 1 and 500 μL Wash Buffer 2. During the extraction, large benchtop centrifuge was used to filter the samples. The RNA was finally eluted by using nuclease-free water instead of Buffer AVE. All the RNA extracts were aliquoted and kept at −80° C. until use.

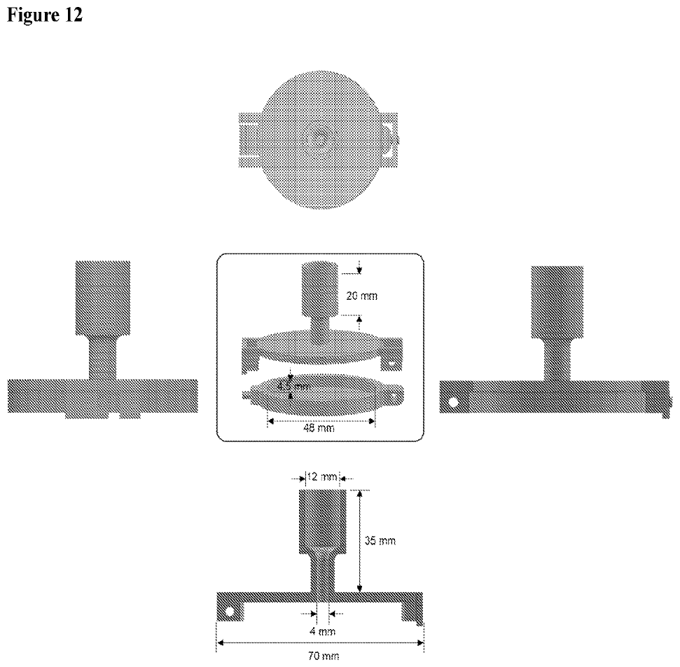

3D-printed Device: The 3D-printed device for pH-paper-based RNA extraction consists of the lid and the container, as shown in FIG. 12. The lid is designed with a sample inlet in which the top diameter is 12 mm, the bottom diameter is 4 mm, and the height is 35 mm. The container is designed with 48 mm diameter and 4.5 mm thickness. The lid and the container are connected using a screw. Prior to pH-paper-based extraction, the lid is covered by a parafilm with a 4-mm middle hole to tightly fit the inner surface and the sample inlet. The high definition Stereolithographic (SLA) laser-based 3D printer Form 2 from Form labs was used to print the device.

pH-paper-based Visual Isothermal Amplification: In this study, RT-DAMP was used as the isothermal amplification method. Table 1 shows the sequence information of the six RT-DAMP primers including FO, RO, FI, RI, FC, and RC (SEQ ID NO: 1-6). These primers were well designed according to the principles previously reported by our lab [X. Ding, Z. Xu, K. Yin, M. Sfeir, C. Liu, Analytical chemistry 2019, 91, 12852]. To develop pH-paper-based visual detection, a non-buffered solution (2×) was first prepared by combining 20 mM (NH₄)₂SO₄, 0.2% (v/v) Tween™ 20, 100 mM KCl, 8 mM KOH, 2.8 mM dNTP, and 16 mM MgSO₄ in a total 500 μL volume. A typical 20-μL pH-paper-based visual isothermal amplification reaction included 1× non-buffered solution, 0.2 μM FO, 0.2 μM RO, 1.6 μM FI, 1.6 μM RI, 1.6 μM FC, 1.6 μM RC, 0.3 U μL⁻¹ WS RTx, 2.5 ng μL⁻¹ ET SSB, 0.2 M betaine, 1.2 U μL⁻¹ Bst 2.0 WS, and the inserted pH paper. For real-time fluorescence detection without pH paper, 0.8× EvaGreen™ and 2.5 μL template solution were added. The prepared reactions were then subjected to incubation at 63° C. for 40 min in the CFX96 Touch Real-Time PCR Detection System™ (Bio-Rad, USA) or the handheld incubator, smart cup.

TABLE 1
The list of all used primers and probes in this study

| Item | Sequence (5′-3′) |
|---|---|
| RT-DAMP forward outer primer | GGCTTCTACGCAGAAGGGA (SEQ ID NO: 1) |
| (FO) targeting SARS-CoV-2 N | |
| gene | |
| RT-DAMP reverse outer primer | TCTGTCAAGCAGCAGCAAAG (SEQ ID NO: 2) |
| (RO) targeting SARS-CoV-2 N | |
| gene | |
| RT-DAMP forward inner primer | ACTGTTGCGACTACGTGATGATTTTGTCAAGCCTCTT |
| (FI) targeting SARS-CoV-2 N | CTCGTTCC (SEQ ID NO: 3) |
| gene | |
| RT-DAMP reverse inner primer | GGAACTTCTCCTGCTAGAATGGCTTTTCAAGAGCAGC |
| (RI) targeting SARS-CoV-2 N | ATCACCGC (SEQ ID NO: 4) |
| gene | |
| RT-DAMP forward competition | ACTGTTGCGACTACGTGATGA (SEQ ID NO: 5) |
| primer (FC) targeting SARS- | |
| CoV-2 N gene | |
| RT-DAMP reverse competition | GGAACTTCTCCTGCTAGAATGGC (SEQ ID NO: 6) |
| primer (RC) targeting SARS- | |
| CoV-2 N gene | |
| nCoV_N1 RT-qPCR forward | GACCCCAAAATCAGCGAAAT (SEQ ID NO: 7) |
| primer | |
| nCoV_N1 RT-qPCR reverse | TCTGGTTACTGCCAGTTGAATCTG (SEQ ID NO: 8) |
| primer | |
| nCoV_N1 RT-qPCR probe | FAM-ACCCCGCATTACGTTTGGTGGACC-BHQ1 |
| | (SEQ ID NO: 9) |

Instrument-free SARS-CoV-2 Rapid Detection: 30 clinical NP swab samples were first treated for 5 min using the lysis buffer (Buffer AVL) before RNA extraction. Then, the lysed samples were put into the 3D-printed device for pH-paper extraction. After less than one-minute extraction, the pH paper was peeled off and directly inserted into the isothermal amplification solution described above, followed by a 40-min incubation at 63° C. in the smart cup. The result was immediately judged based on the color change of pH paper. Thus, the instrument-free SARS-CoV-2 rapid detection could finish the whole assay within 46 min from sample to answer. As for 33 contrived saliva samples, they were prepared by spiking 33 RNA extracts from clinical NP samples into human saliva in a volume ratio of 1:9. These saliva samples were then subjected to the similar processes as those for the clinical NP swabs.

Real-time fluorescence RT-qPCR Assay: The RT-qCPR assay by targeting N gene to detect SARS-CoV-2 was strictly conducted according to the U.S. CDC-released instructions titled CDC 2019-Novel Coronavirus (2019-nCoV) Real-Time RT-PCR Diagnostic Panel (https://www.fda.gov/media/134922/download). GoTaq™ Probe 1-Step RT-qPCR kit recommend by U.S. CDC was used to prepare the reaction mix. A typical 15-μL RT-qPCR reaction included 1× GoTaq™ Probe Master Mix, 0.5 μM nCOV_N1 forward primer, 0.5 μM nCOV_N1 reverse primer, 0.125 μM nCOV_N1 probe, 0.3 μL of the GoScript Reverse Transcriptase Mix™, and 1.0 μL of the target solution. The thermal cycling program consisted of Stage 1 (2.0 min at 25° C.), Stage 2 (15.0 min at 50° C.), Stage 3 (2.0 min at 95° C.) and Stage 4 (45 cycles of 3.0 s at 95° C. and 30 s at 55° C.). The capture point of fluorescence was set at 55° C. in Stage 4. Real-time fluorescence detection was carried out in the Bio-Rad CFX96 Touch Real-Time PCR Detection System™.

Contrived Saliva Samples Preparation: Since current sample collection for COVID-19 diagnostics most involves NP swabs, we have to contrive the saliva samples by spiking heat-inactivated SARS-CoV-2 control and the RNA extracts from clinical NP swabs. The heat-inactivated SARS-CoV-2 control is a commercial product from Twist Bioscience. To prepare saliva with heat-inactivated SARS-CoV-2 control, 4 μL of the control were mixed with 36 μL human saliva, followed by lysate or heat treatment. To prepare saliva with clinical RNA extracts, 30 μL of each RNA extract were mixed with 120 μL human saliva. Then, 28 μL of the saliva sample was used for pH-paper-based RNA extraction, and 140 μL was applied for spin column-based RNA extraction. All the saliva samples could be tested immediately or stored at −80° C. until use.

Colorimetric RT-DAMP Using Cresol Red: The cresol red was dissolved to 50 mM using nuclease-free water. A 2× non-buffered solution was prepared by combining 20 mM (NH₄)₂SO₄, 0.2% (v/v) Tween® 20, 100 mM KCl, 8 mM KOH, 2.8 mM dNTP, and 16 mM MgSO₄ in a total 500 μL volume. A typical 20-μL colorimetric RT-DAMP reaction using cresol red included 1× non-buffered solution, 0.2 μM FO, 0.2 μM RO, 1.6 μM FI, 1.6 μM RI, 1.6 μM FC, 1.6 μM RC, 0.3 U μL⁻¹ WS RTx, 1.2 U μL⁻¹ Bst 2.0 WS, 0.1 mM cresol red, and 1.0 μL target solution. The reactions were incubated at 63° C. for 40 min in Bio-Rad CFX96 Touch Real-Time PCR Detection System™. The sequences of the used primers are shown in Table 1.

### Example 1: Nucleic Acid Extraction

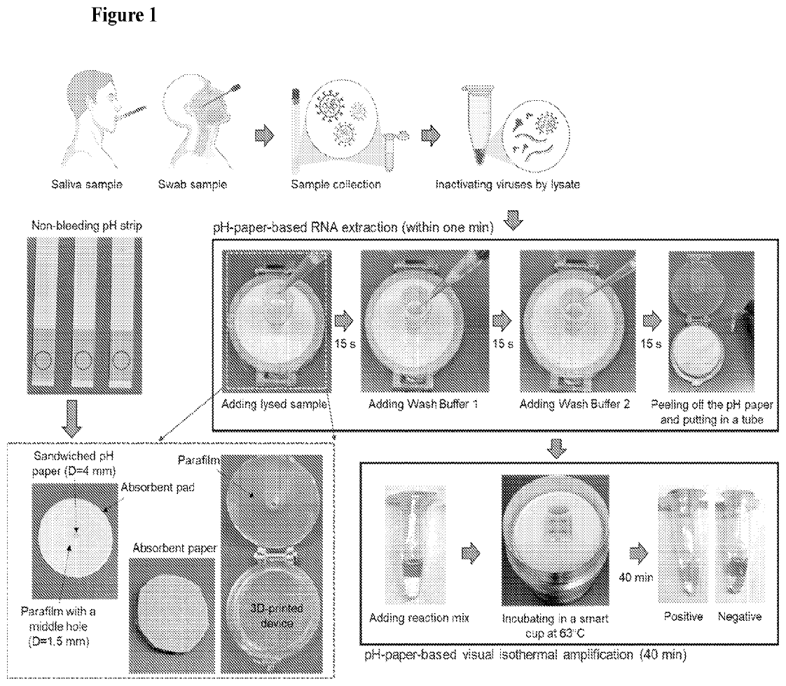

In this study, non-bleeding pH paper as a functional material to achieve both RNA extraction and visual isothermal amplification was employed. The non-bleeding pH paper can provide a random matrix of cellulose fibers to extract and purify RNA from a lysed sample, meanwhile not contaminating or inhibiting the following isothermal amplification. Due to strong capillary force generated by absorbent paper in a 3D-printed device, pH-paper-based RNA extraction can be finished within one minute. After RNA extraction, the pH paper is compatible with isothermal amplification reaction to mediate visual detection without adding any dyes. Accordingly, we develop an instrument-free SARS-CoV-2 rapid detection for onsite COVID-19 diagnostics (FIG. 1). In less than 46 min from saliva samples to answers, pH-paper-based extraction and visual detection (termed pH-EVD) is able to detect 1200 genome equivalent per microliter (GE μL⁻¹) of heat-inactivated SARS-CoV-2, which is comparable to TaqMan probe-based RT-qPCR assay released by U.S. CDC. Through harnessing a handheld incubator, smart cup, our pH-EVD-based instrument-free SARS-CoV-2 rapid detection is also validated by testing 30 clinical nasopharyngeal (NP) swab samples and 33 saliva samples spiked with RNA extracts from clinical NP swabs.

Non-bleeding pH paper is a commercially available material for pH testing. Unlike common pH paper, indicator dyes are covalently bound to this paper to prevent from bleeding and contaminating the samples. Since the pH paper is made of wood cellulose, its random matrix of cellulose fibers can support the extraction of nucleic acids from samples.

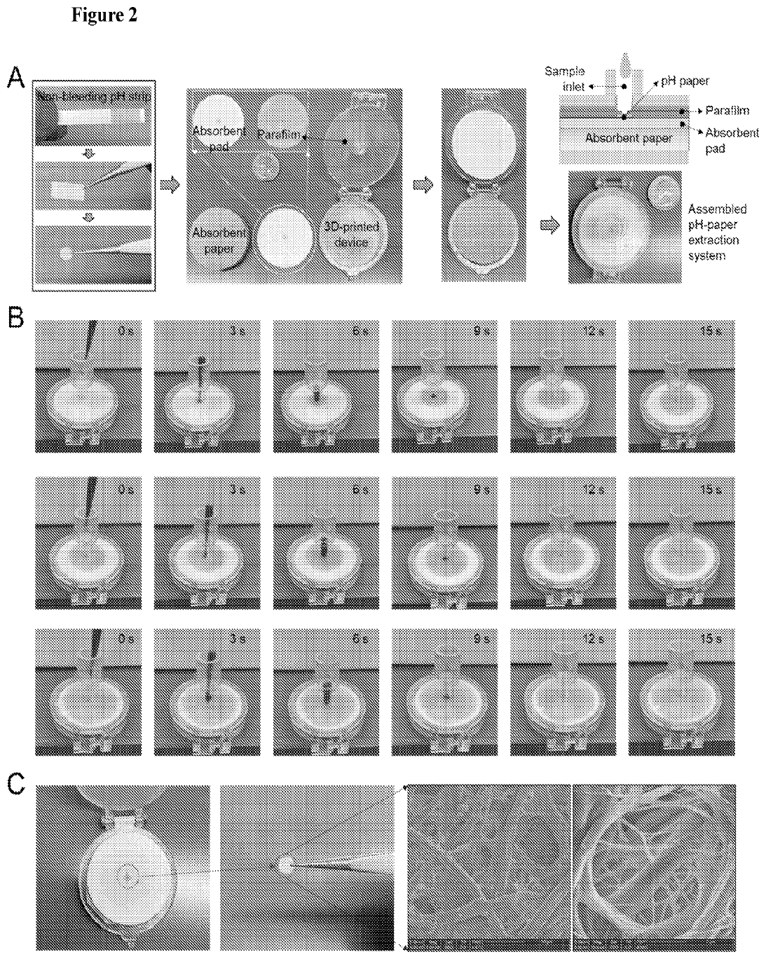

As shown in FIG. 2A, a non-bleeding pH strip is scraped and punched out. Then, an absorbent pad and a parafilm layer are used to sandwich the punched pH paper, followed by stitching. A 3D-printed device filled with absorbent paper is harnessed to assembly the pH-paper extraction system. Viewing from the vertical section of the assembled system (FIG. 2A), pH paper is located at the center and aligns the sample inlet. Another parafilm layer is attached to the inner surface of the lid to further seal the sandwiched pH paper. In the system, absorbent pad and paper can generate strong capillary force to allow fast liquid flow and parafilm layers provide hydrophobic interfaces to lower residue and make sure the sample passes through the pH paper. As demonstrated in FIG. 2B, due to strong capillary force, three continuous liquid loadings can be fished within 45 seconds. Accordingly, we developed a pH-paper RNA extraction system by replacing the liquids with lysed sample, Wash Buffer 1, and Wash Buffer 2 from a commercial QIAamp™ Viral RNA Mini Kit (FIG. 1). FIG. 2C shows that the SEM images of RNA-binding pH paper after extraction. RNA strands adhere to the matrix of cellulose fibers.

### Example 2: Isothermal Amplification and pH Detection

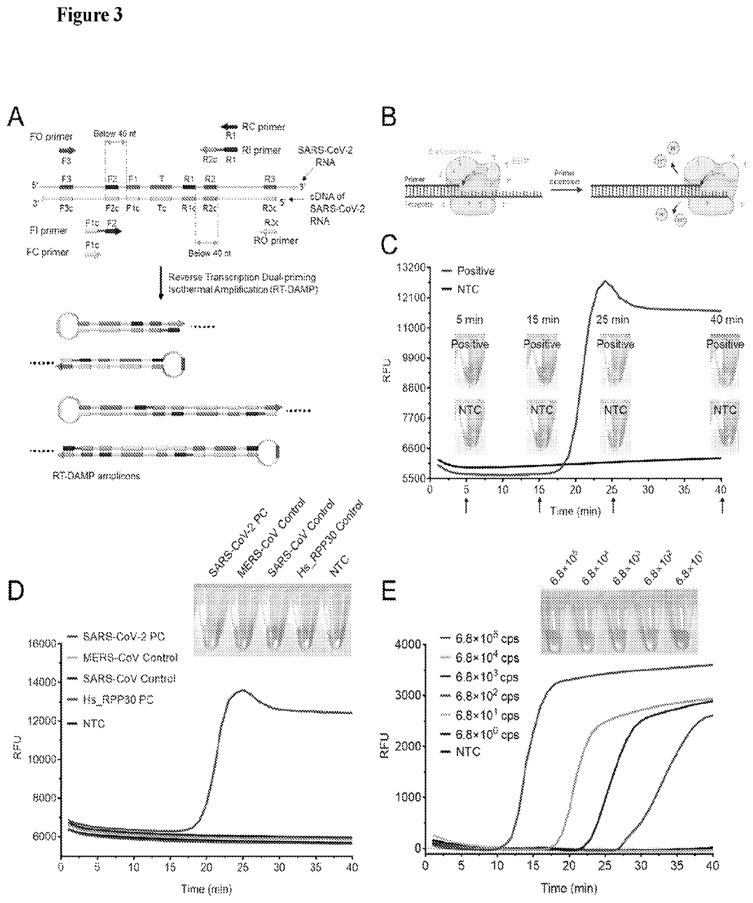

In this study, previously developed RT-DAMP, an isothermal amplification was used [X. Dinget al., Analytical chemistry 2019, 91, 12852]. As shown in FIG. 3A, six primers of RT-DAMP specifically recognize seven distinct sites in the target RNA and its cDNA, and the main amplicons are multiple double-stranded DNAs with closed loops. In a non-buffered RT-DAMP solution, hydrogen ions (H⁺) are generated and accumulated to decrease the pH, attributed to the incorporation of nucleotides during the primer extension by DNA polymerase (FIG. 3B). To prepare the non-buffered solution, Tris-HCl is typically removed to permit the pH change, while KOH is used to tune an alkaline condition (pH around at 8.5) for the initiation of amplification. Therefore, by directly inserting pH paper into the RT-DAMP solution, we successfully developed a simple visual isothermal amplification. As shown in FIG. 3C, pH paper clearly presents yellow (appears light grey in the black and white figures) in positive reactions with targets in as short as 25 min, whereas keeping green brown (appears dark grey in the black and white figures) in no-template control (NTC) reactions. Further, the result based on color change is in accord with the result of real-time fluorescence detection.

Next, the specificity of pH-paper-based visual isothermal amplification using a commercial plasmid control with SARS-CoV-2 N gene sequence from Integrated DNA Technologies (IDT) was assessed. As shown in FIG. 3D, after 40 min incubation, yellow (appears light grey in the black and white figures) is only observed in the reaction with the SARS-CoV-2 positive control, verifying that the visual assay possesses high specificity. In addition, we investigated the sensitivity by testing synthetic SARS-CoV-2 RNA controls from Twist Bioscience (Twist). As depicted in FIG. 3E, pH-paper-based visual RT-DAMP is able to detect down to 680 copies SARS-CoV-2 RNA within 40 min, showing a similar sensitivity as real-time fluorescence RT-DAMP detection. Thus, pH-paper-based visual isothermal amplification has a high specificity and sensitivity for SARS-CoV-2 detection.

Most visual isothermal amplification assays are realized by adding dye indicators or nucleic acid stains. However, these additives, especially the metal ions-chelated indicators (e.g., calcein-Mn) and DNA stains (e.g., SYBR® Green I), could negatively influence the amplification performance. Accordingly, pH-paper-based visual assay is an entirely dye-free format to circumvent the side effect of dyes, thereby mediating high specificity and sensitivity.

### Example 3: pH-Paper-Based RNA Extraction for Visual Detection

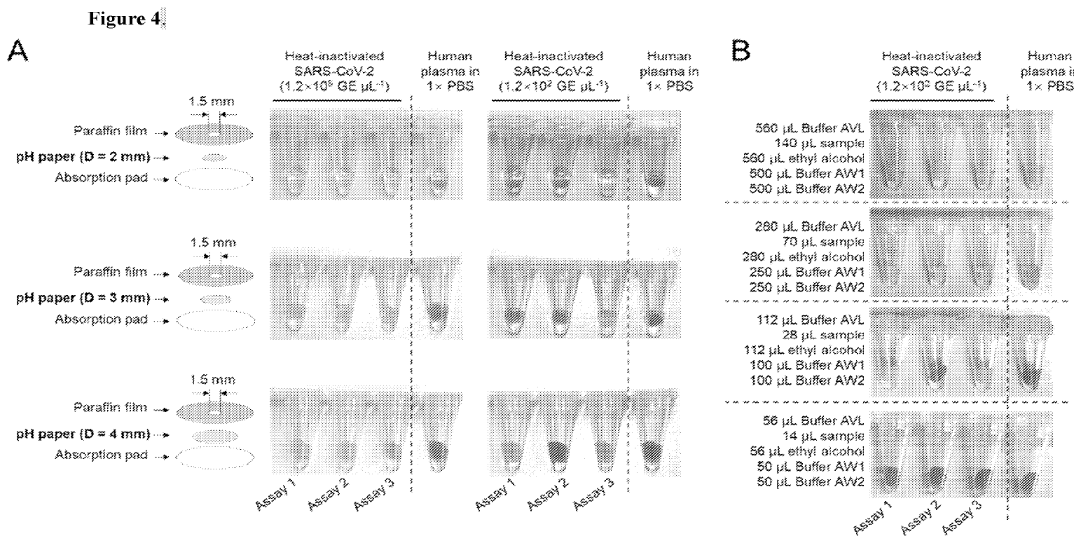

In the pH-paper-based RNA extraction system, a punch of pH paper and commercial RNA extraction reagents are employed. To achieve better detection performance, the diameter of pH paper and the amounts of extraction reagents were varied, followed by visual detection. As shown in FIG. 4A, the larger diameter of pH paper used, the higher sensitivity of visual detection. This improvement is likely attributed to the enlarged filter area provided by the pH paper. However, a pH paper with larger than 4 mm diameter is not applied, since it cannot be immersed entirely by the reaction solution, thereby in turn influencing the visual detection.

By choosing the 4-mm-diameter pH paper, various amounts of extraction reagents in the same ratio of volume according to the instructions of the kit were investigated. FIG. 4B reveals that the combination of 28 μL sample, 112 μL Buffer AVL, 112 μL absolute ethyl alcohol, 112 μL Buffer AW1, and 112 μL Buffer AW2 gives the best performance. Adding more extraction reagents can lead to false positive results, likely due to excessive residues on the pH paper which disturb the initial pH of reaction solution.

### Example 4: Performance of pH-EVD for SARS-CoV-2 Detection

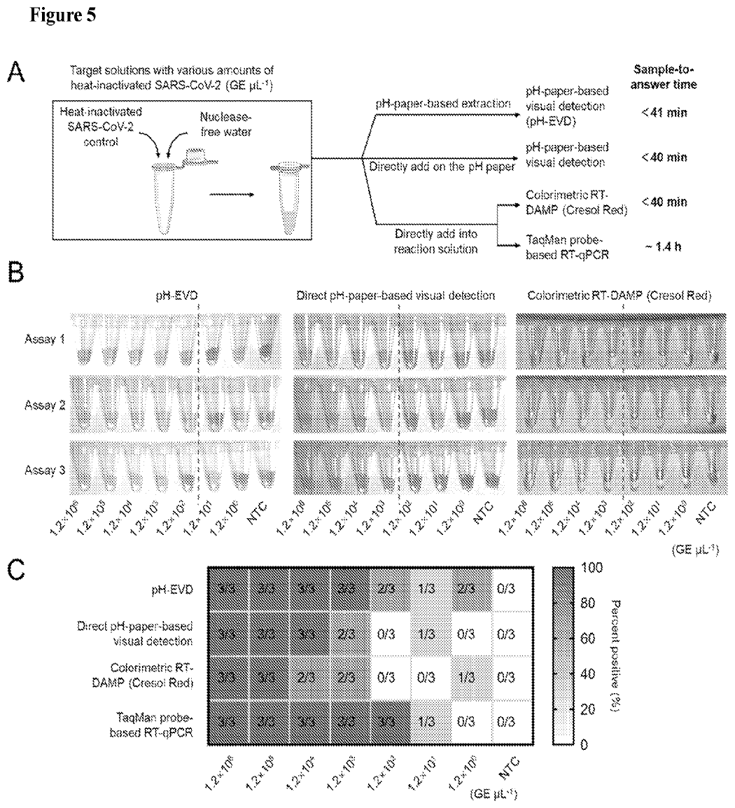

To evaluate the detection performance of pH-EVD, the panel of heat-inactivated SARS-CoV-2 control from BEI Resources was prepared in serial 10-fold dilution from 1.2×10⁶ to 1.2×10⁰ GE μL⁻¹. In addition, direct pH-paper-based visual detection without RNA extraction, extraction-free colorimetric RT-DAMP using cresol red dye, and extraction-free TaqMan™ probe-based RT-qPCR was set-up as parallel experiments (FIG. 5A).

As shown in FIG. 5B, the pH-EVD is able to 100% (3/3) detect 1.2×10³ GE μL⁻¹ of heat-inactivated SARS-CoV-2 in the target solutions, and has 66.7%(⅔), 33.3%(⅓), 66.7% (⅔) probabilities to detect 1.2×10², 1.2×10¹, and 1.2×10⁰ GE μL⁻¹, respectively. However, if removing the RNA extraction, there's only 66.7% (⅔) probability to detect 1.2×10³ GE μL⁻¹ for both pH-based and dye-based visual detections. Also, the color change by pH-EVD is much better than that without extraction, meaning that unpurified samples could interfere with the initial pH of reaction solutions and weaken the performance.

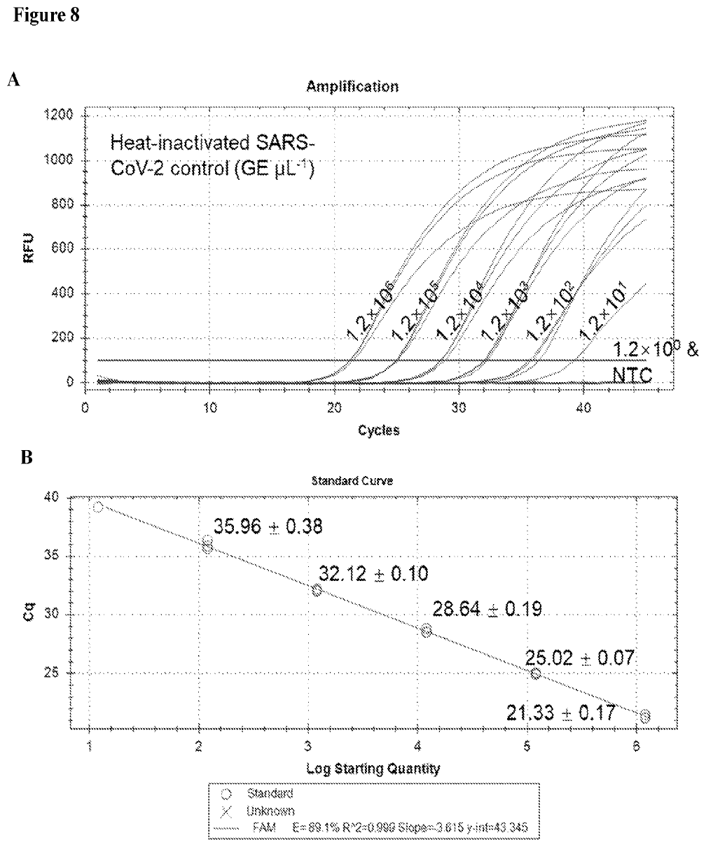

In contrast, extraction-free RT-qPCR has higher performance, since it can 100% (3/3) detect 1.2×10² GE μL⁻¹ SARS-CoV-2 (FIG. 5C). Whereas, the averaged Cq is 35.96 (FIG. 8), already close to the cutoff (Cq=40). In addition, the extraction-free RT-qPCR assay still needs long testing time (˜1.4 h) from sample to answer (FIG. 5A) and greatly depends on large detection instrument. Given this, the pH-EVD assay with less than 41 min sample-to-answer time is more advantageous towards onsite testing.

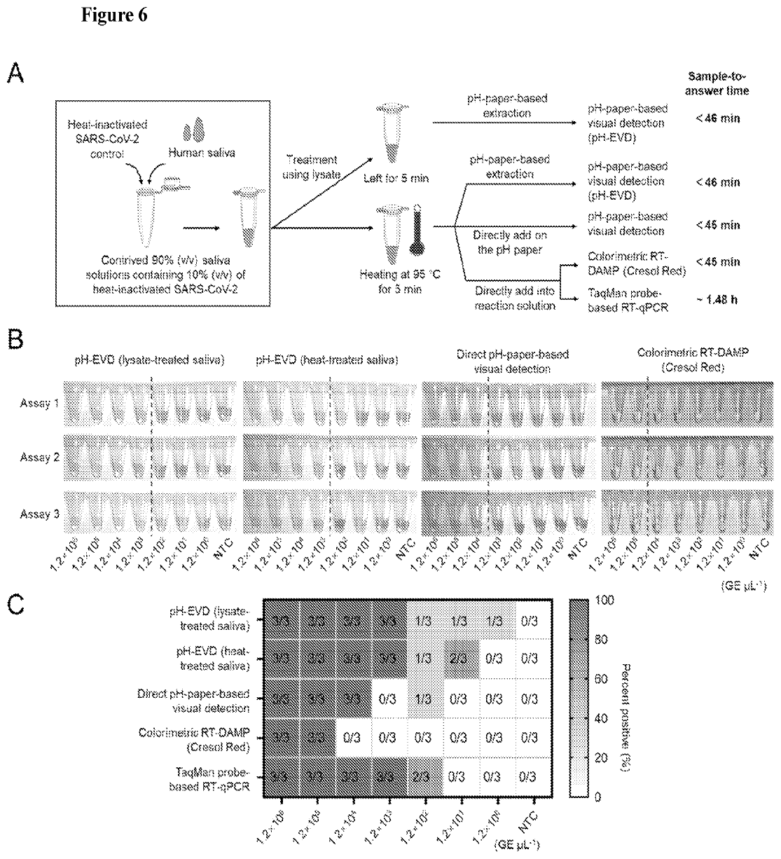

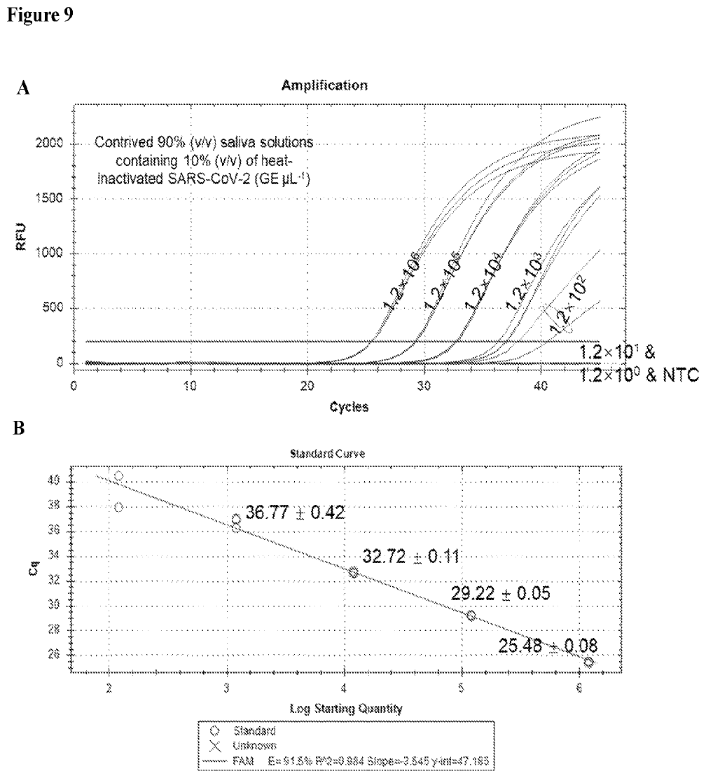

90% (v/v) saliva solutions by spiking 10% (v/v) of the same SARS-CoV-2 controls to further demonstrate the pH-EVD's performance was contrived. Prior to pH-based RNA extraction or direct addition, the contrived saliva solutions were treated by lysis buffer (Buffer AVL) or heated at 95° C. for 5 min (FIG. 6A). In less than 46 min sample-to-answer time, pH-EVD is able to 100% (3/3) detect 1.2×10³ GE μL⁻¹ SARS-CoV-2 from saliva solutions treated by both lysate and heat, showing the same sensitivity as that of assaying serial dilutions of heat-inactivated SARS-CoV-2 control (FIGS. 5B and 6B). While RT-qPCR is able to stably detect 1.2×10³ GE μL⁻¹ SARS-CoV-2 in saliva, the averaged Cq is 36.77 (FIG. 9), higher than that without saliva (its averaged Cq=32.12 in FIG. 8). In addition, a decreased sensitivity from 1.2×10² to 1.2×10³ GE μL⁻¹ is shown in the RT-qPCR assay. Thus, it's obvious that the performance of RT-qPCR is also influenced by heat-treated saliva.

Conclusively, the pH-EVD is robust to identify SARS-CoV-2, not affected by saliva. This promoted visual detection performance benefits from the pH-paper-based extraction to pre-concentrate and purify RNA from lysed- and heat-treated samples.

### Example 5: Clinical Validation of pH-EVD-Based Instrument-Free SARS-CoV-2 Detection

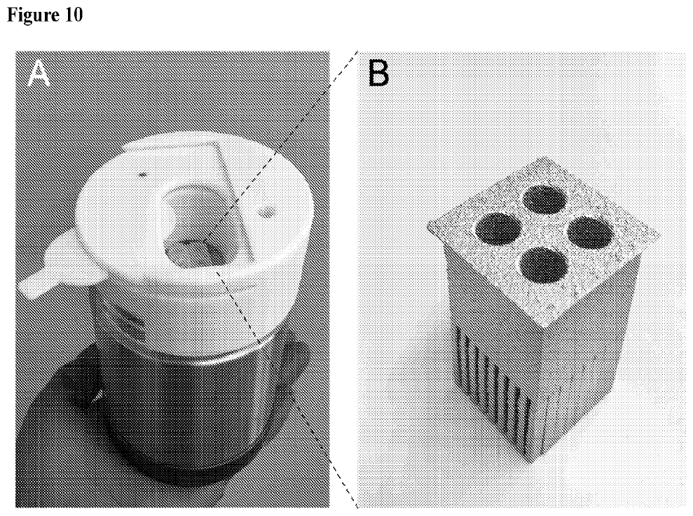

Considering the high performance, pH-EVD was applied to developing an instrument-free SARS-CoV-2 rapid detection by coupling a handheld incubator, smart cup. The smart cup is a minimally-instrumented onsite molecular diagnostic device in which exothermic chemical reaction triggered by water provides heat and a phase-change material regulates the temperature at 63° C. for isothermal amplification (FIG. 10).

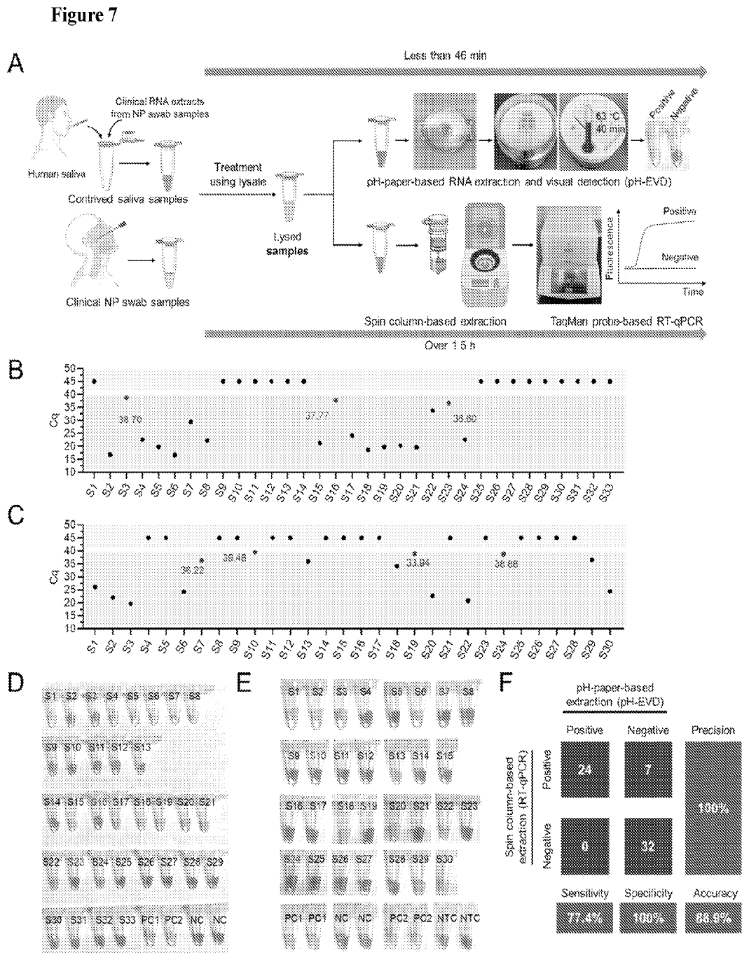

As shown in FIG. 7A, clinical NP swab samples were collected and treated using lysis buffer. Then, the lysed samples were subjected to the pH-EVD assay. Meanwhile, a routine COVID-19 detection approach, real-time fluorescence RT-qPCR assay following spin column-based RNA extraction was used for comparison. Due to current shortage of genuine saliva samples in our facility, we have to contrive them by spiking clinical RNA extracts from NP samples into human saliva. The instrument-free SARS-CoV-2 rapid detection can finish the whole assay from lysing samples to getting results within 46 min, suitable for onsite diagnostics.

FIGS. 7B and 7C display the Cq values for contrived saliva samples (n=33) and clinical NP swab samples (n=30) by RT-qPCR, respectively. Per as the cutoff (Cq=40) suggested by U.S. CDC, 31 samples are tested to be positives (17 for saliva and 14 for NP swab), and 32 samples are negatives (16 for both saliva and NP swab). FIGS. 7D and 7E shows the visual detection results of pH-EVD on testing the saliva and NP swab samples, respectively. By contrast, most of the pH-EVD's results conform with those of RT-qPCR, except the saliva samples of 3, 16, 23, and the NP swabs of 7, 10, 19, 24. Regarding their Cq values from 36.22 to 39.48, very close to the cutoff, these seven samples are supposed to be very weak positives. By taking spin column-based extraction and RT-qPCR as the standards, the confusion matrix indicates that the pH-EVD assay possesses 77.4% sensitivity, 100% specificity, and 100% precision (FIG. 7F).

1. A method of detecting a nucleic acid of interest comprising:
a. amplifying by isothermal amplification a nucleic acid of interest present in a nucleic acid sample bound in cellulose fibers of a pH sensitive element to generate an amplification product, wherein the isothermal amplification is performed in the absence of a pH buffer, and wherein the isothermal amplification of the nucleic acid of interest results in the release of hydrogen ions on the pH sensitive element; and
b. identifying a pH detected by the pH sensitive element, wherein a pH of 7.5 or less indicates the release of hydrogen ions during the isothermal amplification and the presence of the nucleic acid of interest in the nucleic acid sample

2. The method of claim 1, wherein the prior to the amplifying, the method comprises obtaining the nucleic acid sample from a biological sample, wherein the obtaining comprises lysing cells present in the biological sample to generate a processed biological sample, and contacting the processed biological sample with the pH sensitive element to bind the nucleic acid sample to the cellulose fibers of the pH sensitive element.

3. The method of claim 2, wherein the lysing comprises treating the biological sample with a lysin or exposing the biological sample to heat.

4. The method of claim 2, wherein the contacting step comprises filtering the processed biological sample through the pH sensitive element, wherein the nucleic acid sample from the processed biological sample binds to the pH sensitive element.

5. The method of claim 4, wherein the filtering comprises filtering the biological sample through the pH sensitive element by capillary force.

6. (canceled)

7. The method of claim 2, wherein the lysing, contacting, or filtering comprises one or more wash step(s) and wherein the one or more wash steps occurs prior to the isothermal amplification.

8. The method of claim 1, wherein a pH of between 6 and 7.5 indicates the presence of the nucleic acid of interest in the nucleic acid sample and/or a pH of more than 7.5 indicates the absence of the nucleic acid of interest in the nucleic acid sample.

9. (canceled)

10. The method of claim 1, wherein the nucleic acid sample and/or the nucleic acid of interest is RNA or DNA.

11. The method of claim 1, wherein the pH sensitive element is non-bleeding pH paper and/or comprises colorimetric detection.

12. (canceled)

13. The method of claim 1, wherein the isothermal amplification comprises dual-priming mediated isothermal amplification (DAMP), reverse transcription DAMP (RT-DAMP), loop-mediated isothermal amplification (LAMP), nucleic acid sequence based amplification (NASBA), recombinase polymerase amplification (RPA), helicase dependent amplification (HDA), rolling circle amplification (RCA), strand-displacement amplification (SDA), exponential amplification reaction (EXPAR), or a combination thereof.

14. (canceled)

15. The method of claim 13, wherein the DAMP or RT-DAMP comprises:
a. contacting a nucleic acid sample suspected of containing the target nucleic acid with primers; and
b. cyclically amplifying the target nucleic acid to provide amplified target nucleic acid, wherein the primers comprise a forward outer primer (FO), a reverse outer primer (RO), a forward inner primer (FI), a reverse inner primer (RI), a forward reverse pairing-competition primer (FC), a reverse pairing-competition primer (RC) or a combination thereof.

16. (canceled)

17. The method of claim 1, where in the biological sample is saliva, plasma, urine, whole blood, nasal mucus or a combination thereof.

18. The method of claim 1, wherein the isothermal amplification is performed in solution, cellulose fiber matrix, or a combination thereof.

19. The method of claim 18, wherein the pH of the solution is more than 8.0 prior to the isothermal amplification.

20.-21. (canceled)

22. The method of claim 1, wherein the nucleic acid of interest is a nucleic acid specific for a pathogenic organism.

23. The method of claim 1, wherein the pathogenic organism is SARS-CoV-2, human immunodeficiency virus (HIV), tuberculosis (TB), ebola, meningitis, influenza A, influenza B, human papilloma virus (HPV), hepatitis C virus (HCV), hepatitis B virus (HBV), or a combination thereof.

24. (canceled)

25. The method of claim 1, wherein the isothermal amplification is carried out at between 550 and 700 and/or for between 30 and 60 minutes.

26. (canceled)

27. The method of claim 1, wherein the methods can detect at least 1,000 genome equivalent per microliter.

28. A device comprising:
a housing having a top portion and a bottom portion, wherein the top portion of the housing includes a first through-hole;
a fluid inlet extending vertically from a top surface of the top portion of the housing, wherein the fluid inlet is in fluid communication with the first through-hole;
a non-absorbent layer positioned adjacent a bottom surface of the top portion of the housing, wherein the non-absorbent layer includes a second through-hole vertically aligned with the first through-hole;
one or more absorbent layers positioned between the non-absorbent layer and the bottom portion of the housing;
wherein the one or more absorbent layers and the non-absorbent layer are positioned to accommodate placement of a pH sensitive element positioned between the one or more absorbent layers and the non-absorbent layer; and
wherein a pH sensitive element positioned between the one or more absorbent layers and the non-absorbent layer is vertically aligned with both the first through-hole and the second through-hole.

29.-35. (canceled)

36. A kit comprising two or more of:
a. a pH sensitive element
b. primers specific for a nucleic acid of interest
c. reagents for performing isothermal amplification in the absence of a pH buffer, and
d. means of warming the isothermal amplification reagents.

37.-42. (canceled)


SEQUENCE LISTING (9 sequences)

SEQ ID NO: 1 - DNA, 19 bp, synthetic construct
  source 1..19: mol_type = other DNA
ggcttctacgcagaaggga

SEQ ID NO: 2 - DNA, 20 bp, synthetic construct
  source 1..20: mol_type = other DNA
tctgtcaagcagcagcaaag

SEQ ID NO: 3 - DNA, 45 bp, synthetic construct
  source 1..45: mol_type = other DNA
actgttgcgactacgtgatgattttgtcaagcctcttctcgttcc

SEQ ID NO: 4 - DNA, 45 bp, synthetic construct
  source 1..45: mol_type = other DNA
ggaacttctcctgctagaatggcttttcaagagcagcatcaccgc

SEQ ID NO: 5 - DNA, 21 bp, synthetic construct
  source 1..21: mol_type = other DNA
actgttgcgactacgtgatga

SEQ ID NO: 6 - DNA, 23 bp, synthetic construct
  source 1..23: mol_type = other DNA
ggaacttctcctgctagaatggc

SEQ ID NO: 7 - DNA, 20 bp, synthetic construct
  source 1..20: mol_type = other DNA
gaccccaaaatcagcgaaat

SEQ ID NO: 8 - DNA, 24 bp, synthetic construct
  source 1..24: mol_type = other DNA
tctggttactgccagttgaatctg

SEQ ID NO: 9 - DNA, 24 bp, synthetic construct
  source 1..24: mol_type = other DNA
  modified_base 1: mod_base = OTHER; note = modified with a FAM
  modified_base 24: mod_base = OTHER; note = modified with BHQ1
accccgcattacgtttggtggacc